(19) United States
(12) Patent Application Publication
(10) Pub. No.: US 2022/0308028 A1
(43) Pub. Date: Sep. 29, 2022

(54) DETERMINATION APPARATUS, DETERMINATION SYSTEM, DETERMINATION METHOD, AND COMPUTER READABLE MEDIUM

(71) Applicant: Asahi Kasei Microdevices Corporation, Tokyo (JP)

(72) Inventors: Takaaki FURUYA, Tokyo (JP); Keiichiro KUWATA, Tokyo (JP)

(21) Appl. No.: 17/702,774

(22) Filed: Mar. 23, 2022

(30) Foreign Application Priority Data
Mar. 26, 2021 (JP) 2021-053521
Mar. 11, 2022 (JP) 2022-037716

(51) Int. Cl. G01N 33/00; G08B 21/18; G08B 25/10

(52) U.S. Cl. CPC G01N 33/004; G01N 33/0063; G08B 21/182; G08B 25/10

(57) ABSTRACT

Provided is a determination apparatus including: a determination unit configured to determine, based on a carbon dioxide concentration in a determination target, a situation of the carbon dioxide concentration in the determination target and a measurement situation of the carbon dioxide concentration in the determination target; a notification unit configured to notify a determination result by the determination unit; an information acquisition unit configured to acquire improvement information according to at least one of a situation of the carbon dioxide concentration and a measurement situation of the carbon dioxide concentration; and a control unit configured to control the notification unit to have the notification unit notify the determination result and the improvement information.

The contents of the following Japanese patent application(s) are incorporated herein by reference:

NO. 2021-053521 filed in JP on Mar. 26, 2021

NO. 2022-037716 filed in JP on Mar. 11, 2022

### BACKGROUND

### 1. TECHNICAL FIELD

The present invention relates to a determination apparatus, a determination system, determination method, and a computer readable medium.

### 2. RELATED ART

In Patent Literature 1, it is described that “from a carbon dioxide sensor installed in a target facility and a biological sensor provided in a sleeping place in the target facility, a carbon dioxide concentration in the target facility and a biological signal of a subject in the sleeping place are respectively acquired” (paragraph 0006).

### LIST OF CITED REFERENCES

### Patent Literatures

[Patent Literature 1] Japanese Unexamined Patent Application, Publication No. 2020-071621

The above summary of the invention does not enumerate all of the features of the invention. Subcombinations of these feature groups may also be inventive.

### BRIEF DESCRIPTION OF THE DRAWINGS

FIG. 1 is a block diagram illustrating one example of a determination system 200 according to one embodiment of the present invention.

FIG. 2 is a block diagram illustrating another example of the determination system 200 according to one embodiment of the present invention.

FIG. 3 is a block diagram illustrating another example of the determination system 200 according to one embodiment of the present invention.

FIG. 4 is a block diagram illustrating another example of the determination system 200 according to one embodiment of the present invention.

FIG. 5 is a block diagram illustrating another example of the determination system 200 according to one embodiment of the present invention.

FIG. 6 is a block diagram illustrating another example of the determination system 200 according to one embodiment of the present invention.

FIG. 7 is a block diagram illustrating another example of the determination system 200 according to one embodiment of the present invention.

FIG. 8 is a block diagram illustrating another example of the determination system 200 according to one embodiment of the present invention.

FIG. 9 is a block diagram illustrating another example of the determination system 200 according to one embodiment of the present invention.

FIG. 10 is a block diagram illustrating one example of a determination apparatus 100 according to one embodiment of the present invention.

FIG. 11 is a block diagram illustrating another example of the determination apparatus 100 according to one embodiment of the present invention.

FIG. 12 is a block diagram illustrating another example of the determination system 200 according to one embodiment of the present invention.

FIG. 13 is a block diagram illustrating another example of the determination system 200 according to one embodiment of the present invention.

FIG. 14 is a block diagram illustrating another example of the determination system 200 according to one embodiment of the present invention.

FIG. 15 is a flowchart illustrating one example of a determination method according to one embodiment of the present invention.

FIG. 16 is a flowchart illustrating another example of the determination method according to one embodiment of the present invention.

FIG. 17 is a flowchart illustrating another example of the determination method according to one embodiment of the present invention.

FIG. 18 is a flowchart illustrating another example of the determination method according to one embodiment of the present invention.

FIG. 19 is a flowchart illustrating another example of the determination method according to one embodiment of the present invention.

FIG. 20 is a flowchart illustrating another example of the determination method according to one embodiment of the present invention.

FIG. 21 illustrates one example of a computer 2200 in which the determination apparatus 100 according to embodiments of the present invention may be entirely or partially embodied.

### DESCRIPTION OF EXEMPLARY EMBODIMENTS

Hereinafter, the present invention will be described by way of embodiments of the invention, but the following embodiments are not intended to restrict the invention according to the claims. In addition, not all combinations of features described in the embodiments necessarily have to be essential to solving means of the invention.

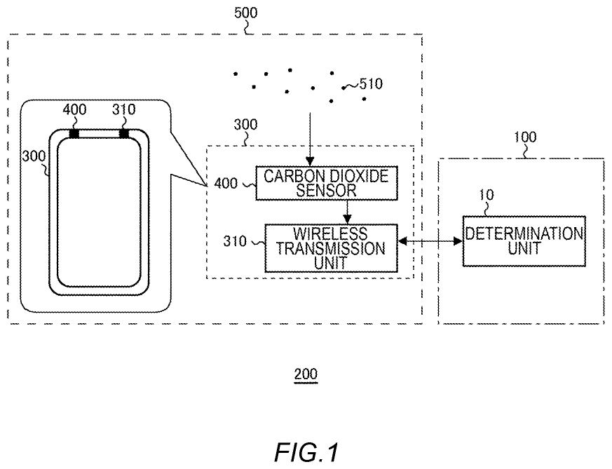

FIG. 1 is a block diagram illustrating one example of a determination system 200 according to one embodiment of the present invention. The determination system 200 includes a determination apparatus 100 and a mobile terminal 300. The mobile terminal 300 has a CO₂ (carbon dioxide) sensor 400. The mobile terminal 300 is, for example, a smartphone, a tablet, a head mounted display (HMD), a wearable device, or the like. The wearable device refers to a computer mounted to a part of a body such as an arm, a leg, or a head region. The wearable device is, for example, a smartwatch.

The CO₂ (carbon dioxide) sensor 400 is configured to measure a CO₂ (carbon dioxide) concentration in a determination target 500. The determination target 500 is a target object that is a determination target related to the CO₂ (carbon dioxide) concentration. The target object is, for example, a room in doors. The target object may be a predetermined space in open air. The CO₂ (carbon dioxide) sensor 400 measures a concentration of CO₂ (carbon dioxide) 510 contained in the air present in the target object.

When the determination target 500 is a room in doors, the mobile terminal 300 may be arranged inside the room. When the determination target 500 is a room in doors, the determination apparatus 100 may be arranged inside the room or may also be arranged outside the room.

In the present example, the mobile terminal 300 includes a wireless transmission unit 310. In the present example, the wireless transmission unit 310 is configured to transmit the concentration of the CO₂ (carbon dioxide) 510 measured by the CO₂ (carbon dioxide) sensor 400 to the determination apparatus 100.

The determination apparatus 100 includes a determination unit 10. The determination unit 10 is configured to determine, based on the CO₂ (carbon dioxide) concentration in the determination target 500, a situation of the CO₂ (carbon dioxide) concentration in the determination target 500 and a measurement situation of the CO₂ (carbon dioxide) concentration in the determination target 500. In the present example, the CO₂ (carbon dioxide) concentration in the determination target 500 is transmitted by the wireless transmission unit 310.

The determination unit 10 may be a central processing unit (CPU). The determination apparatus 100 may be a computer including the CPU, a memory, an interface, and the like. The determination apparatus 100 may also be a portable computer such as a tablet.

The situation of the CO₂ (carbon dioxide) concentration in the determination target 500 is set as a situation Sd. The measurement situation of the CO₂ (carbon dioxide) concentration in the determination target 500 is set as a measurement situation Sm. The situation Sd refers to at least one of an extent of the concentration and an extent of a concentration change of the CO₂ (carbon dioxide) 510 in the determination target 500. The situation Sd refers to, for example, whether the CO₂ (carbon dioxide) concentration in the determination target 500 is an abnormal value, is a normal value close to the abnormal value, rapidly approaches the abnormal value, or the like.

The measurement situation Sm refers to a situation related to a measurement environment of the concentration of the CO₂ (carbon dioxide) 510 in the determination target 500. The measurement situation Sm refers to a situation such as, for example, whether the concentration of the CO₂ (carbon dioxide) 510 in the determination target 500 is measured in a situation where human breath is in contact with the CO₂ (carbon dioxide) sensor 400 or is measured in a situation where the CO₂ (carbon dioxide) sensor 400 is arranged in a location close to a wall, whether the mobile terminal 300 having the CO₂ (carbon dioxide) sensor 400 is on the move, or the like.

The determination unit 10 may output a determination result of the situation Sd and the measurement situation Sm. The determination result is set as a determination result Rd. With this configuration, a user of the determination apparatus 100 can find out the situation Sd and the measurement situation Sm of the concentration of the CO₂ (carbon dioxide) 510 in the determination target 500.

The determination unit 10 may determine the situation Sd of the concentration of the CO₂ (carbon dioxide) 510 based on a change over time of the concentration of the CO₂ (carbon dioxide) 510. Since the situation Sd is determined based on the change over time of the concentration of the CO₂ (carbon dioxide) 510, the user of the determination apparatus 100 can find out the situation Sd determined based on the change over time of the concentration of the CO₂ (carbon dioxide) 510 and the measurement situation Sm of the concentration of the CO₂ (carbon dioxide) 510. If the concentration of CO₂ (carbon dioxide) 510 is increasing with time change, the determination unit 10 may determine that the situation Sd is in an abnormal state even if the concentration of CO₂ (carbon dioxide) 510 is below a certain reference value. Time changes in the concentration of CO₂ (carbon dioxide) 510 may include the time derivative, time integral, statistical distribution, statistic, time mean, variance per hour, deviation from the mean per hour, and predicted values of the concentration of CO₂ (carbon dioxide) 510 after a certain time that are predicted from the time changes.

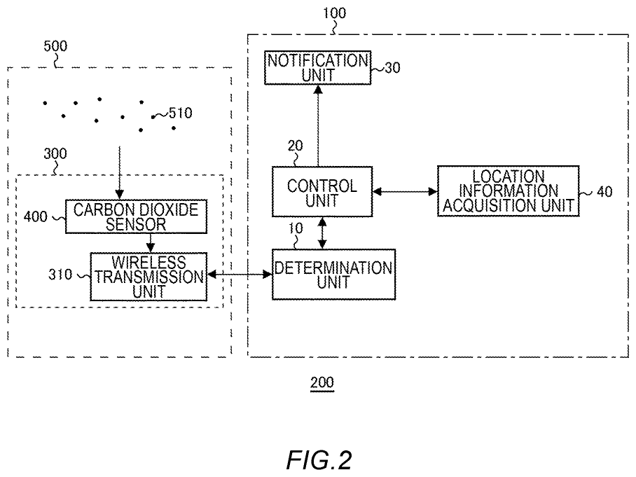

FIG. 2 is a block diagram illustrating another example of the determination system 200 according to one embodiment of the present invention. In the present example, the determination apparatus 100 further includes a control unit 20, a notification unit 30, and a location information acquisition unit 40. The determination system 200 of the present example is different from the determination system 200 illustrated in FIG. 1 in the above described aspect.

The notification unit 30 is configured to notify the determination result Rd by the determination unit 10. The notification unit 30 may notify the determination result Rd in a form that can be perceived by a human visual sense. The notification unit 30 is, for example, a display, a monitor, or the like. The location information acquisition unit 40 is configured to acquire location information of the determination target 500. The location information acquisition unit 40 is, for example, a global positioning system (GPS). When the determination apparatus 100 is a portable computer such as a tablet, the notification unit 30 may be a display of the computer, and the location information acquisition unit 40 may be the GPS included in the computer. The location information acquisition unit 40 may also acquire location information of the determination apparatus 100.

The control unit 20 is configured to control, based on the location information of the determination target 500 which is acquired by the location information acquisition unit 40, whether the determination result Rd by the determination unit 10 is to be notified by the notification unit 30. The control unit 20 may control whether the determination result Rd is to be notified by the notification unit 30. The control unit 20 may be a central processing unit (CPU). The determination unit 10 and the control unit 20 may be a single CPU.

When the location information of the determination target 500 which is acquired by the location information acquisition unit 40 indicates, for example, a vicinity of a factory that causes combustion of a fossil fuel, the concentration of the CO₂ (carbon dioxide) 510 in the vicinity of the factory may be steadily high as compared with a location other than the vicinity of the factory. In the situation Sd of the CO₂ (carbon dioxide) concentration which is previously expected as described above where a response to the situation Sd is not necessary, a configuration may be adopted where the control unit 20 does not notify the determination result Rd by the determination unit 10 on the notification unit 30.

The determination unit 10 may also determine the measurement situation Sm of the concentration of the CO₂ (carbon dioxide) 510 in the determination target 500 based on the location information of the determination target 500 which is acquired by the location information acquisition unit 40. The determination unit 10 may also determine the measurement situation Sm based on the concentration of the CO₂ (carbon dioxide) 510 and the location information of the determination target 500. The determination unit 10 may also determine the measurement situation Sm not based on the concentration of the CO₂ (carbon dioxide) 510, but based on the location information of the determination target 500.

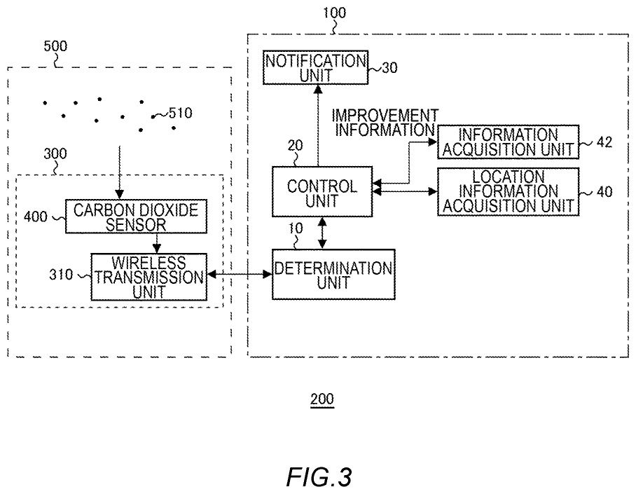

FIG. 3 is a block diagram illustrating another example of the determination system 200 according to one embodiment of the present invention. In the present example, the determination apparatus 100 further includes an information acquisition unit 42. The determination system 200 of the present example is different from the determination system 200 illustrated in FIG. 2 in the above described aspect. The information acquisition unit 42 is configured to acquire improvement information according to at least one of the situation Sd of the carbon dioxide concentration and the measurement situation Sm of the carbon dioxide concentration. The improvement information is set as improvement information Si.

In the present example, the improvement information Si according to the situation Sd refers to advertisement information or the like of a contractor who can improve or eliminate a factor causing the situation Sd. When the situation Sd is, for example, a situation where the concentration of the CO₂ (carbon dioxide) 510 is an abnormal value, the contractor who can improve or eliminate the factor is a ventilation contractor who can eliminate the abnormal value. When the situation Sd is, for example, a situation where the concentration of the CO₂ (carbon dioxide) 510 is an abnormal value, the improvement information Si may include improvement information for eliminating the abnormal value, maintenance information, or the like.

In the present example, the improvement information Si according to the measurement situation Sm refers to advertisement information or the like of a contractor who can remedy the measurement situation Sm. When the measurement situation Sm is, for example, a situation where the CO₂ (carbon dioxide) sensor 400 performs the measurement in an insufficiently calibrated state, a contractor who can remedy the measurement situation Sm is a calibration contractor who can eliminate the insufficiently calibrated state.

In the present example, the control unit 20 controls the notification unit 30 in such a way as to notify the determination result Rd by the determination unit 10 and the improvement information Si on the notification unit 30 based on the location information of the determination target 500. Therefore, when the situation Sd is, for example, a situation where the concentration of the CO₂ (carbon dioxide) 510 is an abnormal value, the control unit 20 can notify the advertisement information or the like of one or more contractors who are close to the location of the determination target 500 and who can eliminate the situation where the concentration of the CO₂ (carbon dioxide) 510 is an abnormal value on the notification unit 30. With this configuration, the user of the determination apparatus 100 can find out the contractor who can eliminate the situation where the concentration of the CO₂ (carbon dioxide) 510 is an abnormal value. When having the notification unit 30 notify the determination result Rd and improvement information Si, the notification unit 30 may notify a plurality of contractors. The order in which the plurality of contractors are notified or the size of the notification or the like, may be prioritized. For example, the order of the above a contractor to be notified may change for each carbon dioxide concentration range, and certain a contractor may be notified at a higher priority.

The control unit 20 may change the determination result Rd and the improvement information Si which are to be notified by the notification unit 30 based on the location information of the determination target 500. The control unit 20 may control the notification unit 30 in such a way as to notify the determination result Rd and the improvement information Si which have been changed on the notification unit 30. When the determination apparatus 100 is, for example, a portable tablet or the like, the location information of the determination apparatus 100 changes along with the movement of the determination apparatus 100. Therefore, for example, the contractor who can eliminate the situation where the concentration of the CO₂ (carbon dioxide) 510 is an abnormal value and who is close to the current location of the determination target 500 may change along with the movement of the determination apparatus 100. When the contractor who is close to the current location of the determination target 500 changes along with the movement of the determination apparatus 100, the control unit 20 may change the determination result Rd and the improvement information Si to be notified by the notification unit 30 according to the location information of the determination target 500.

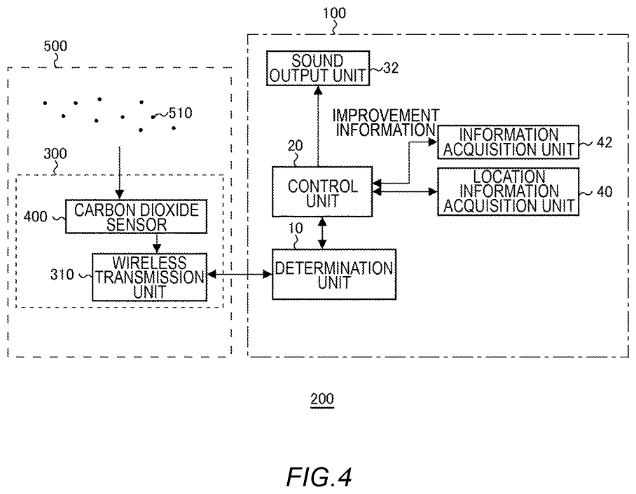

FIG. 4 is a block diagram illustrating another example of the determination system 200 according to one embodiment of the present invention. In the present example, the determination apparatus 100 includes a sound output unit 32 instead of the notification unit 30. The determination system 200 of the present example is different from the determination system 200 illustrated in FIG. 3 in the above described aspect. The sound output unit 32 is configured to output a sound related to the determination result Rd by the determination unit 10. The sound output unit 32 may output the determination result Rd in a form that can be perceived by a human hearing sense. The sound output unit 32 is, for example, a speaker. The notification unit 30 may be an optical notification unit that notifies with light or a vibration notification unit that notifies with vibration.

In the present example, the control unit 20 controls the sound output unit 32 in such a way as to cause the sound output unit 32 to output a sound related to the determination result Rd by the determination unit 10 and the improvement information Si based on the location information of the determination target 500. The sound related to the determination result Rd and the improvement information Si is, for example, a name or the like of a contractor who can eliminate the situation where the concentration of the CO₂ (carbon dioxide) 510 is an abnormal value.

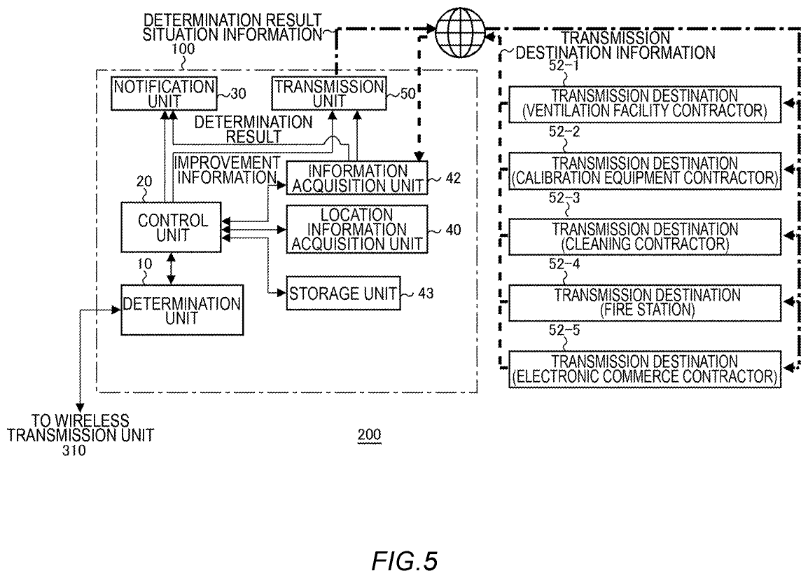

FIG. 5 is a block diagram illustrating another example of the determination system 200 according to one embodiment of the present invention. In FIG. 5, the determination target 500 and the mobile terminal 300 which are illustrated in FIG. 2 to FIG. 4 are omitted. In the present example, the determination apparatus 100 further includes a transmission unit 50. The determination system 200 of the present example is different from the determination system 200 illustrated in FIG. 3 in the above described aspect. The transmission unit 50 is configured to transmit the determination result Rd and the improvement information Si. The transmission unit 50 may wirelessly transmit the determination result Rd and the improvement information Si.

The information acquisition unit 42 may further acquire transmission destination information regarding a transmission destination to which the determination result Rd and the improvement information Si are to be transmitted. The transmission destination is set as a transmission destination 52. The transmission destination information is set as transmission destination information Is. The transmission destination 52 may be at least one of the contractor who can eliminate the factor causing the situation Sd of the CO₂ (carbon dioxide) concentration in the determination target 500 and the contractor who can remedy the measurement situation Sm of the CO₂ (carbon dioxide) concentration in the determination target 500.

The information acquisition unit 42 may acquire the transmission destination information Is regarding a plurality of the transmission destinations 52. The information acquisition unit 42 may acquire the transmission destination information Is through an Internet line. In the present example, the information acquisition unit 42 acquires the transmission destination information Is regarding at least one of five transmission destinations 52 (transmission destination 52-1 to transmission destination 52-5). The information acquisition unit 42 may wirelessly acquire the transmission destination information Is. In FIG. 5, acquisition routes of the transmission destination information Is are represent by bolded broken arrows.

In the present example, the transmission destination 52-1 is a ventilation facility contractor, the transmission destination 52-2 is a calibration equipment contractor, the transmission destination 52-3 is a clearing contractor, the transmission destination 52-4 is a fire station, and the transmission destination 52-5 is an electronic commerce contractor. The ventilation facility contractor is, for example, a manufacturing contractor of an air conditioning facility. When the determination target 500 is a room in doors, the clearing contractor is, for example, a clearing contractor for a duct for discharging indoor air to an outdoor space. The electronic commerce contractor is, for example, a contractor who sells a product via the Internet. These transmission destinations are examples. The transmission destination 52 may be, for example, a contractor operating an application such as air quality management, or it may be an insurance company.

The control unit 20 may control the transmission unit 50 in such a way as to transmit the determination result Rd and the improvement information Si to the transmission destination 52 based on the location information of the determination target 500. The determination result Rd and the improvement information Si may be transmitted to the transmission destination 52 through the Internet line. In FIG. 5, transmission routes of the determination result Rd and the improvement information Si are represented by bolded dashed-dotted arrows.

In the present example, the determination result Rd and the improvement information Si are transmitted based on the location information of the determination target 500. Therefore, when the situation Sd is, for example, a situation where the concentration of the CO₂ (carbon dioxide) 510 is an abnormal value, the control unit 20 can control the transmission unit 50 in such a way as to transmit the determination result Rd and the improvement information Si to the contractor who can eliminate the situation where the concentration of the CO₂ (carbon dioxide) 510 is an abnormal value and who is the closest to the current location of the determination target 500. With this configuration, the closest contractor can more quickly arrive at the determination target 500 than a contractor farther from the current location of the determination target than the closest contractor.

The control unit 20 may control the transmission unit 50 in such a way as to transmit the determination result Rd and the improvement information Si to the transmission destination 52 further based on the determination result Rd and the improvement information Si, and the transmission destination information Is. The control unit 20 may select the transmission destination 52 to which the determination result Rd and the improvement information Si are to be transmitted from among the plurality of transmission destinations 52 based on the determination result Rd and the improvement information Si, and the transmission destination information Is. When the situation Sd is, for example, a situation where the concentration of the CO₂ (carbon dioxide) 510 is an abnormal value, the control unit 20 may control the transmission unit 50 in such a way as to transmit the determination result Rd and the improvement information Si to at least one of the transmission destination 52-1 (in the present example, the ventilation facility contractor) and the transmission destination 52-3 (in the present example, the clearing contractor). When the measurement situation Sm is, for example, a situation where the CO₂ (carbon dioxide) sensor 400 performs the measurement in an insufficiently calibrated state, the control unit 20 may control the transmission unit 50 in such a way as to transmit the determination result Rd and the improvement information Si to the transmission destination 52-2 (in the present example, the calibration equipment contractor).

It should be noted that the determination apparatus 100 may further include a storage unit 43, or it may be sufficient without the storage unit 43. In the present example, the determination apparatus 100 includes the storage unit 43. When the determination apparatus 100 includes the storage unit 43, the storage unit 43 may store the transmission destination information Is acquired by the information acquisition unit 42. The storage unit 43 may store at least one of the determination result Rd and the improvement information Si. When the determination apparatus 100 does not include the storage unit 43, at least one of the transmission destination information Is, the determination result Rd, and the improvement information Si may be stored in a server or a cloud server arranged outside the determination apparatus 100.

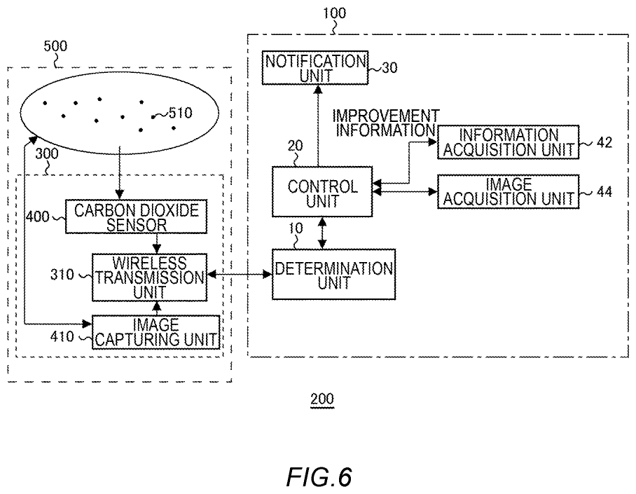

FIG. 6 is a block diagram illustrating another example of the determination system 200 according to one embodiment of the present invention. In the present example, the determination apparatus 100 does not include the location information acquisition unit 40, but further includes an image acquisition unit 44. The determination system 200 of the present example is different from the determination system 200 illustrated in FIG. 3 in the above described aspect. The image acquisition unit 44 is configured to acquire an image of the determination target 500.

The mobile terminal 300 may include an image capturing unit 410 configured to capture an image of the determination target 500. The image capturing unit 410 is, for example, a camera. When the mobile terminal 300 is a smartphone, the image capturing unit 410 may be a camera built in the smartphone. The wireless transmission unit 310 may transmit the image of the determination target 500 which is captured by the image capturing unit 410 to the determination unit 10. The image acquisition unit 44 may acquire the image of the determination target 500 which is transmitted to the determination unit 10.

The control unit 20 may control the notification unit 30 in such a way as to notify the determination result Rd and the improvement information Si on the notification unit 30. In the present example, the control unit 20 controls the notification unit 30 in such a way as to notify the determination result Rd and the improvement information Si on the notification unit 30 based on the image of the determination target 500 which is acquired by the image acquisition unit 44. In the present example, the improvement information Si includes, for example, warning information, advertisement information of the transmission destination 52 (see FIG. 5), or the like. The warning information is, for example, alert information indicating that the CO₂ (carbon dioxide) concentration in the determination target 500 which is measured by the CO₂ (carbon dioxide) sensor 400 exceeds a predetermined concentration. The advertisement information of the transmission destination 52 (see FIG. 5) is, for example, advertisement information of a ventilation facility contractor, advertisement information of a calibration facility contractor, or the like.

In the present example, the determination result Rd and the improvement information Si are notified by the notification unit 30 based on the image of the determination target 500. Therefore, the user of the determination apparatus 100 can find out the determination result Rd and the improvement information Si by visually recognizing the notification unit 30.

The determination unit 10 may correct the determination result Rd based on an image acquired by the image acquisition unit 44. When the image acquired by the image acquisition unit 44 is an image of equipment that may cause a deviation from a steady emission amount of the CO₂ (carbon dioxide) in the determination target 500, the determination unit 10 may correct the determination result Rd to the determination result Rd in a case where the concentration of the CO₂ (carbon dioxide) 510 measured by the CO₂ (carbon dioxide) sensor 400 is corrected.

When an image of predetermined equipment that emits a predetermined amount or more of CO₂ (carbon dioxide) is acquired by the image acquisition unit 44, the determination unit 10 may correct the determination result Rd. The predetermined amount of CO₂ (carbon dioxide) may refer to a volume or a mass of CO₂ (carbon dioxide) emitted per unit time. A case where the predetermined amount or more of CO₂ (carbon dioxide) is emitted is a case where the emission amount of CO₂ (carbon dioxide) is not steady such as a case where combustion of a fossil fuel is caused in the determination target 500.

The predetermined equipment that emits the predetermined amount or more of CO₂ (carbon dioxide) may refer to equipment having a high probability that the predetermined amount or more of CO₂ (carbon dioxide) is to be emitted. The equipment is, for example, equipment that causes combustion of a fossil fuel. The equipment is, for example, a space heater. When the image acquired by the image acquisition unit 44 is an image of the equipment that causes the combustion of the fossil fuel, the equipment has a high probability that the emission amount of CO₂ (carbon dioxide) per unit time is higher than that of equipment that does not cause the combustion of the fossil fuel. Therefore, when the image acquired by the image acquisition unit 44 is, for example, an image of the equipment that causes the combustion of the fossil fuel, the determination unit 10 may correct the determination result Rd to the determination result Rd indicating a concentration higher than the concentration of the CO₂ (carbon dioxide) 510 measured by the CO₂ (carbon dioxide) sensor 400.

The determination unit 10 may also determine the measurement situation Sm of the concentration of the CO₂ (carbon dioxide) 510 in the determination target 500 based on the image acquired by the image acquisition unit 44. The determination unit 10 may also determine the measurement situation Sm based on the concentration of the CO₂ (carbon dioxide) 510 and the image acquired by the image acquisition unit 44. The determination unit 10 may also determine the measurement situation Sm not based on the concentration of the CO₂ (carbon dioxide) 510, but based on the image acquired by the image acquisition unit 44.

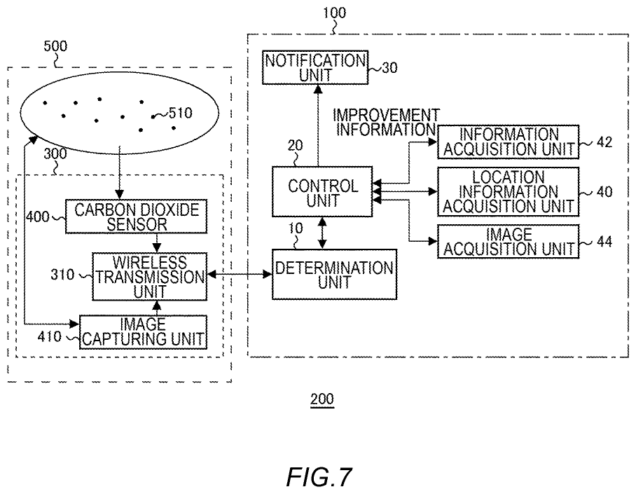

FIG. 7 is a block diagram illustrating another example of the determination system 200 according to one embodiment of the present invention. In the present example, the determination apparatus 100 includes the location information acquisition unit 40. The determination system 200 of the present example is different from the determination system 200 illustrated in FIG. 6 in the above described aspect.

In the present example, the control unit 20 controls the notification unit 30 in such a way as to notify the determination result Rd and the improvement information Si on the notification unit 30 based on the image of the determination target 500 which is acquired by the image acquisition unit 44 and the location information of the determination target 500 which is acquired by the location information acquisition unit 40. Therefore, for example, when the plurality of transmission destinations 52 (see FIG. 5) are present, advertisement information of the contractor closest to the location of the determination target 500 is likely to be notified by the notification unit 30.

When the determination apparatus 100 is a tablet or the like and the determination apparatus 100 is on the move, the contractor closest to the current location of the determination apparatus 100 may change along with the movement of the determination apparatus 100. When the determination apparatus 100 is on the move, the control unit 20 may change the improvement information Si to be notified by the notification unit 30 based on the image of the determination target 500 which is acquired by the image acquisition unit 44 and the location information of the determination apparatus 100.

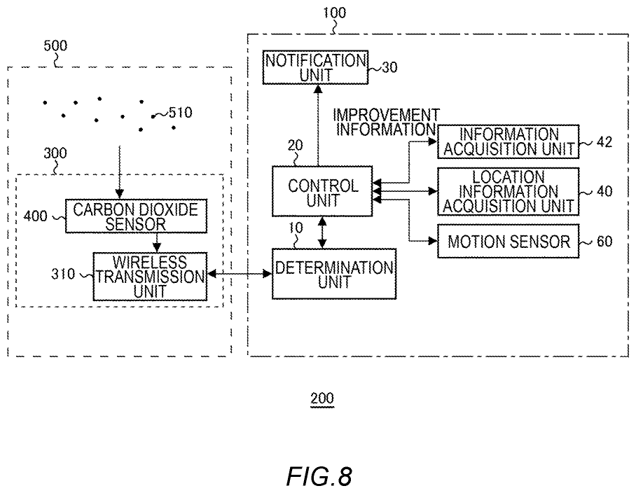

FIG. 8 is a block diagram illustrating another example of the determination system 200 according to one embodiment of the present invention. In the present example, the determination apparatus 100 further includes a motion sensor 60. The determination system 200 of the present example is different from the determination system 200 illustrated in FIG. 3 in the above described aspect. The motion sensor 60 is configured to detect at least one of an acceleration, an angular rate, and terrestrial magnetism information of the determination apparatus 100.

In the present example, the improvement information Si is information based on at least one of the acceleration, the angular rate, and the terrestrial magnetism information of the determination apparatus 100 which are measured by the motion sensor 60. When the determination apparatus 100 is a smartphone, a tablet, or the like, the determination apparatus 100 tends to be in a moving state. When the motion sensor 60 detects the acceleration of the determination apparatus 100, a probability that the determination apparatus 100 is on the move is high. When the determination apparatus 100 is on the move, the user of the determination apparatus 100 may be in a state where it is not possible to visibly recognize the notification unit 30. Therefore, when the motion sensor 60 detects the acceleration of the determination apparatus 100, the control unit 20 may control the notification unit 30 in such a way as to avoid notification of the determination result Rd on the notification unit 30. The control unit 20 may also control the notification unit 30 in such a way as to notify, on the notification unit 30, the improvement information Si according to the measurement situation Sm indicating that the CO₂ (carbon dioxide) concentration is measured under an environment where the determination apparatus 100 is on the move.

When a living body uses the determination apparatus 100, if the motion sensor 60 detects acceleration associated with body movements during sleep of the said living body, the determination apparatus 100 may determine that the said living body is in a sleep state. The determination apparatus 100 may control the notification unit 30 to notify the notification unit 30 of the improvement information Si according to the concentration of CO₂ (carbon dioxide) 510 while the said living body is sleeping. If the motion sensor 60 detects at least one of acceleration, angular velocity, and geomagnetic variation associated with the motion of the said living body in motion, the determination apparatus 100 may determine that the said living body is in motion. The determination apparatus 100 may control the notification unit 30 to notify the notification unit 30 of the improvement information Si according to the concentration of CO₂ (carbon dioxide) 510 during exercise by the said living body.

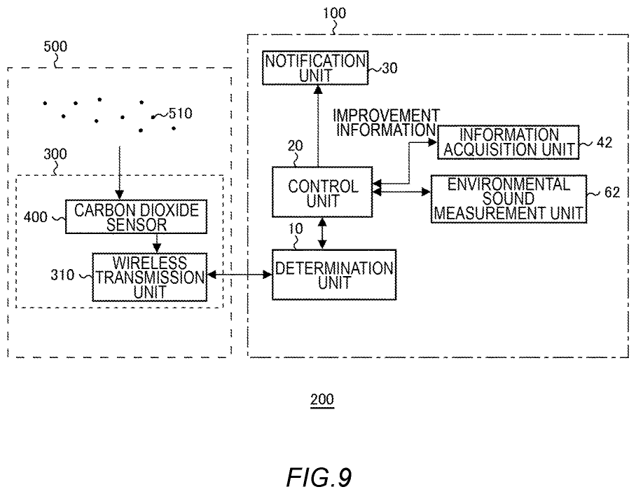

FIG. 9 is a block diagram illustrating another example of the determination system 200 according to one embodiment of the present invention. In the present example, the determination apparatus 100 further includes an environmental sound measurement unit 62 instead of the location information acquisition unit 40 in FIG. 3. The determination system 200 of the present example is different from the determination system 200 illustrated in FIG. 3 in the above described aspect. The environmental sound measurement unit 62 is configured to measure an environmental sound in the determination target 500. The environmental sound measurement unit 62 may measure an amplitude of the environmental sound.

In the present example, the improvement information Si according to the measurement situation Sm of the carbon dioxide concentration is information based on the environmental sound measured by the environmental sound measurement unit 62. When the concentration of the CO₂ (carbon dioxide) 510 in the determination target 500 is measured in a situation where, for example, the CO₂ (carbon dioxide) sensor 400 is arranged in a location at a distance that is shorter than a predetermined distance from a wall, the CO₂ (carbon dioxide) 510 tends to remain in a range under the predetermined distance.

The environmental sound measured by the environmental sound measurement unit 62 is set as an environmental sound S. The environmental sound S heading towards a wall is set as an environmental sound S1, and the environmental sound S reflected by the wall is set as an environmental sound S2. A clock time at which the environmental sound S1 is measured by the environmental sound measurement unit 62 is set as a clock time T1, and a clock time at which the environmental sound S2 is measured by the environmental sound measurement unit 62 is set as a clock time T2. A time period of a difference between the clock time T2 and the clock time T1 is set as a time period ΔT. A sound velocity of the environmental sound S1 and the environmental sound S2 is set as a sound velocity Vs. It should be noted that the environmental sound S2 is a so called reverberant sound.

The control unit 20 can calculate a distance that is twice as long as a distance from the CO₂ (carbon dioxide) sensor 400 to a wall by dividing the time period ΔT by the sound velocity Vs. With this configuration, the control unit 20 can calculate a distance between the wall and the CO₂ (carbon dioxide) sensor 400. It should be noted that the environmental sound S1 may be a sound output by the sound output unit 32 (see FIG. 4). In a case where the environmental sound S1 is the sound output by the sound output unit 32, the clock time T1 may be a clock time at which the environmental sound S1 is output by the sound output unit 32.

The information acquisition unit 42 may acquire the improvement information Si based on the environmental sound measured by the environmental sound measurement unit 62. The control unit 20 may control the notification unit 30 in such a way as to notify the determination result Rd and the improvement information Si on the notification unit 30.

The determination unit 10 may also determine the measurement situation Sm of the concentration of the CO₂ (carbon dioxide) 510 in the determination target 500 based on the environmental sound measured by the environmental sound measurement unit 62. The determination unit 10 may also determine the measurement situation Sm based on the concentration of the CO₂ (carbon dioxide) 510 and the environmental sound measured by the environmental sound measurement unit 62. The determination unit 10 may also determine the measurement situation Sm not based on the concentration of the CO₂ (carbon dioxide) 510, but based on the environmental sound measured by the environmental sound measurement unit 62.

When the living body uses the determination apparatus 100, if the environmental sound measurement unit 62 detects a sound associated with the sound of breathing during sleep of the living body, the determination apparatus 100 may determine that the living body is in a sleep state. The determination apparatus 100 may control the notification unit 30 to notify the notification unit 30 of the improvement information Si according to the concentration of CO₂ (carbon dioxide) 510 while the said living body is sleeping.

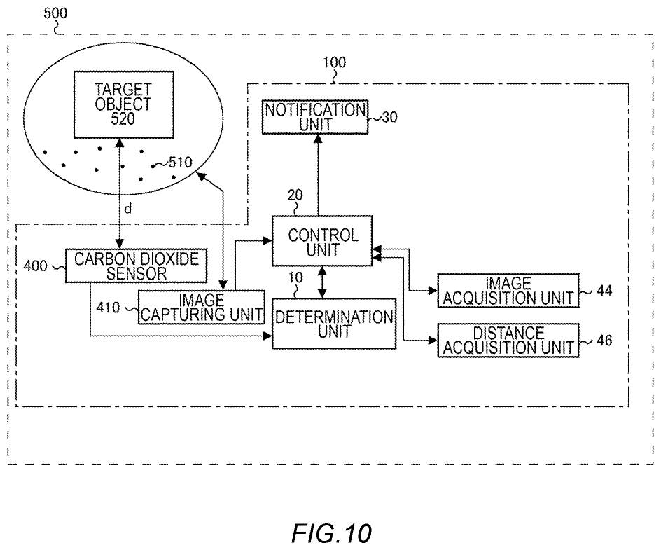

FIG. 10 is a block diagram illustrating one example of the determination apparatus 100 according to one embodiment of the present invention. In the present example, the determination apparatus 100 includes the CO₂ (carbon dioxide) sensor 400. In the present example, the determination apparatus 100 further includes a distance acquisition unit 46. The distance acquisition unit 46 is configured to acquire a distance between the CO₂ (carbon dioxide) sensor 400 and a target object 520. In the present example, the distance acquisition unit 46 acquires the distance between the CO₂ (carbon dioxide) sensor 400 and the target object 520 based on the image acquired by the image acquisition unit 44. The distance is set as a distance d. It should be noted that when the distance acquisition unit 46 is to acquire the distance d not based on the image acquired by the image acquisition unit 44, the distance acquisition unit 46 may acquire the distance d by an electro-optical distance meter or light detection and ranging (LiDAR).

The determination apparatus 100 may include the image capturing unit 410 configured to capture an image of the determination target 500. The image capturing unit 410 is, for example, a camera. When the determination apparatus 100 is a computer such as a tablet, the image capturing unit 410 may be a camera built in the computer. The image acquisition unit 44 may acquire the image of the determination target 500 which is captured by the image capturing unit 410.

The target object 520 is a structural object in the determination target 500 which is a structural object that may affect at least one of the situation Sd and the measurement situation Sm of the CO₂ (carbon dioxide) concentration in the determination target 500. The structural object is, for example, a building. The target object 520 may be a wall, floor, a ceiling, a window, or a door of the structural object or a living body. The living body may be a human body or may also be a body of an animal.

When the distance d acquired by the distance acquisition unit 46 is shorter than a predetermined distance, the control unit 20 may control the notification unit 30 in such a way as to notify warning information on the notification unit 30. The warning information may be warning information indicating that the measurement situation Sm of the concentration of the CO₂ (carbon dioxide) 510 is inappropriate.

When the target object 520 is, for example, a wall, the CO₂ (carbon dioxide) 510 tends to remain in a range under a predetermined distance from the target object 520. For this reason, when the distance d between the CO₂ (carbon dioxide) sensor 400 and the target object 520 (wall) is shorter than the predetermined distance, the determination result Rd in the determination target 500 may be inaccurate. Therefore, when the distance d between the CO₂ (carbon dioxide) sensor 400 and the target object 520 (wall) is shorter than the predetermined distance, the warning information may be notified by the notification unit 30. The predetermined distance is, for example, 50 cm.

When the target object 520 is, for example, a window or a door, a probability is high that two spaces where the concentration of the CO₂ (carbon dioxide) 510 is different from each other are interconnected by the window or the door. For this reason, the concentration of the CO₂ (carbon dioxide) 510 tends to fluctuate in a range under a predetermined distance from the target object 520 (window or door). For this reason, when the distance d between the CO₂ (carbon dioxide) sensor 400 and the target object 520 (window or door) is shorter than the predetermined distance, the determination result Rd in the determination target 500 may be inaccurate. Therefore, when the distance d between the CO₂ (carbon dioxide) sensor 400 and the target object 520 (window or door) is shorter than the predetermined distance, the warning information may be notified by the notification unit 30. The predetermined distance is, for example, 1.5 m.

When the target object 520 is, for example, a living body, since the living body emits CO₂ (carbon dioxide) through breathing, the concentration of the CO₂ (carbon dioxide) 510 tends to be higher in a range under a predetermined distance from the target object 520 (living body) than that in a range at and beyond the predetermined distance. For this reason, the distance d between the CO₂ (carbon dioxide) sensor 400 and the target object 520 (living body) is shorter than the predetermined distance, the determination result Rd in the determination target 500 may be inaccurate. Therefore, when the distance d between the CO₂ (carbon dioxide) sensor 400 and the target object 520 (living body) is shorter than the predetermined distance, the warning information may be notified by the notification unit 30. The predetermined distance is, for example, 1 m from the target object 520 (living body). The predetermined distance may be 2 m from a front of the target object 520 (living body), or may also be 1 m from a side of the target object 520 (living body). When the living body is a person, the front of the living body refers to an orientation in which the person has bilateral symmetry by setting a spine as a center line. The side of the living body refers to an orientation in which the person is rotated by 90 degrees in either left or right direction with respect to the front by setting the spine as the center line.

In the present example, the determination apparatus 100 may include a proximity sensor instead of the CO₂ (carbon dioxide) sensor 400. In the present example, the determination apparatus 100 may include one of the CO₂ (carbon dioxide) sensor 400 and the proximity sensor, and may also include both of those sensors. The proximity sensor can detect that the distance d to the target object 520 is shorter than a predetermined distance. When the target object 520 is, for example, a human body and also the determination apparatus 100 is, for example, a smartphone, the proximity sensor may detect whether a person is in a call. When the person is in a call, since the person may emit more CO₂ (carbon dioxide) as compared with a case where the person is not in a call, the determination result Rd in the determination target 500 may be inaccurate. Therefore, when the distance d between the proximity sensor and the target object 520 is shorter than the predetermined distance, the warning information may be notified by the notification unit 30. The predetermined distance is, for example, 10 cm. It should be noted that the proximity sensor may be an induction type proximity sensor, may also be a capacitive proximity sensor, or may also be a magnetic proximity sensor.

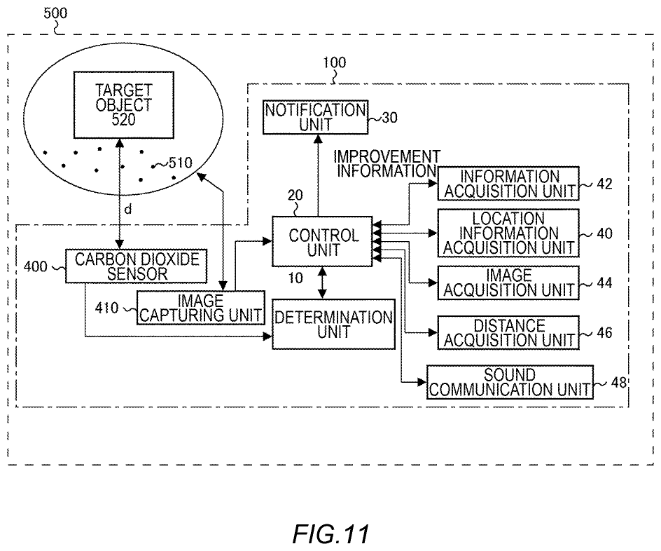

FIG. 11 is a block diagram illustrating another example of the determination apparatus 100 according to one embodiment of the present invention. The determination apparatus 100 of the present example is different from the determination apparatus 100 illustrated in FIG. 10 in an aspect that a sound communication unit 48 is further included. The sound communication unit 48 is configured to communicate a sound. The sound communication unit 48 transmits a sound from the determination apparatus 100 and also receives a sound from another apparatus. The sound communication unit 48 is, for example, a microphone. The other apparatus is, for example, another smartphone. The sound is, for example, a conversation.

In a case where the sound is communicated by the sound communication unit 48, the determination unit 10 may correct the determination result Rd. A case where the sound is communicated is, for example, a case of an active call when the determination apparatus 100 is a smartphone. As described above, when the person is in a call, the person may emit more CO₂ (carbon dioxide) as compared with a case where the person is not in a call. For this reason, the CO₂ (carbon dioxide) sensor 400 tends to be in a state in steady contact with a human breath that is a breath containing CO₂ (carbon dioxide). The human breath is, for example, an expiratory air of a person. Therefore, in a case where the sound is communicated by the sound communication unit 48, the determination unit 10 may correct the determination result Rd to the determination result Rd indicating a concentration lower than the concentration of the CO₂ (carbon dioxide) 510 measured by the CO₂ (carbon dioxide) sensor 400.

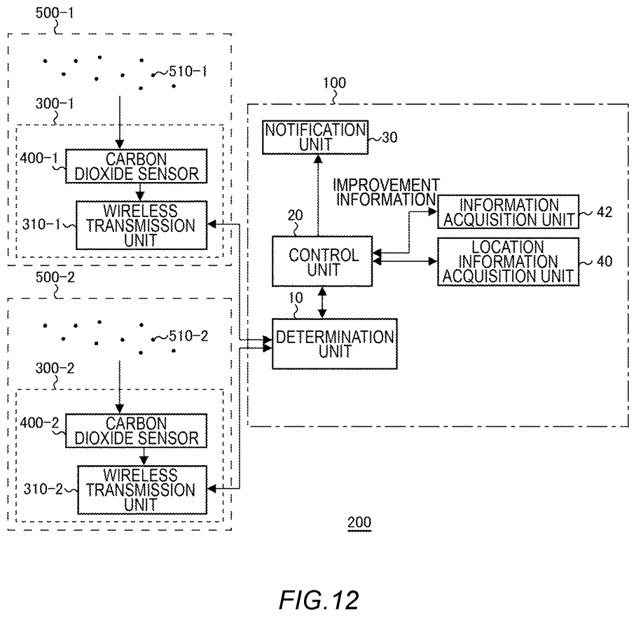

FIG. 12 is a block diagram illustrating another example of the determination system 200 according to one embodiment of the present invention. The determination system 200 may include a plurality of the mobile terminals 300. In the present example, the determination system 200 includes two pieces of the mobile terminals 300 (mobile terminal 300-1 and mobile terminal 300-2). The determination system 200 of the present example is different from the determination system 200 illustrated in FIG. 3 in an aspect that the two mobile terminals 300 are included. In the present example, the mobile terminal 300-1 is set as the first mobile terminal 300-1, and the mobile terminal 300-2 is set as the second mobile terminal 300-2.

Each of the plurality of mobile terminals 300 may include the wireless transmission unit 310 and the CO₂ (carbon dioxide) sensor 400. In the present example, the first mobile terminal 300-1 includes a CO₂ (carbon dioxide) sensor 400-1 and a wireless transmission unit 310-1, and the second mobile terminal 300-2 includes a CO₂ (carbon dioxide) sensor 400-2 and a wireless transmission unit 310-2. The CO₂ (carbon dioxide) sensor 400-1 is configured to measure a CO₂ (carbon dioxide) concentration in a determination target 500-1. The CO₂ (carbon dioxide) sensor 400-2 is configured to measure a CO₂ (carbon dioxide) concentration in a determination target 500-2. The wireless transmission unit 310-1 is configured to transmit a concentration of CO₂ (carbon dioxide) 510-1 measured by the CO₂ (carbon dioxide) sensor 400-1 to the determination apparatus 100. The wireless transmission unit 310-2 is configured to transmit a concentration of CO₂ (carbon dioxide) 510-2 measured by the CO₂ (carbon dioxide) sensor 400-2 to the determination apparatus 100.

The determination unit 10 may determine the situation Sd of the CO₂ (carbon dioxide) concentration based on the concentration of the CO₂ (carbon dioxide) 510 in the determination target 500 and a concentration of CO₂ (carbon dioxide) 510 in another determination target 500 different from the determination target 500. In the present example, the determination unit 10 determines the situation Sd of the CO₂ (carbon dioxide) concentration based on the concentration of the CO₂ (carbon dioxide) 510-1 in the determination target 500-1 and the concentration of the CO₂ (carbon dioxide) 510-2 in the determination target 500-2. The determination target 500-1 and the determination target 500-2 are not interconnected.

In the present example, the determination unit 10 determines the situation Sd of the CO₂ (carbon dioxide) concentration of one of the determination target 500-1 and the determination target 500-2. In the present example, when the determination unit 10 is to determine the situation Sd of the CO₂ (carbon dioxide) concentration in the determination target 500-1, the determination unit 10 compares the CO₂ (carbon dioxide) concentration in the determination target 500-1 with the CO₂ (carbon dioxide) concentration in the determination target 500-2 to determine the situation Sd of the CO₂ (carbon dioxide) concentration in the determination target 500-1. Therefore, the determination unit 10 more easily determines the more accurate situation Sd as compared with a case where the situation Sd of the CO₂ (carbon dioxide) concentration in the single determination target 500 is to be determined.

In the determination system 200 of the present example, the determination apparatus 100 has a mode of the determination apparatus 100 illustrated in FIG. 3, but the determination apparatus 100 may adopt a mode of the determination apparatus 100 illustrated in any of FIG. 1, FIG. 2, and FIG. 4 to FIG. 9.

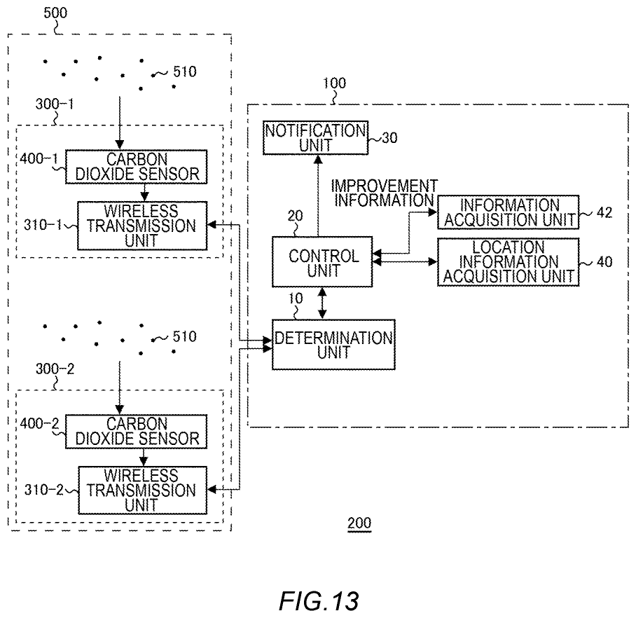

FIG. 13 is a block diagram illustrating another example of the determination system 200 according to one embodiment of the present invention. In the present example, the CO₂ (carbon dioxide) sensor 400-1 of the first mobile terminal 300-1 and the CO₂ (carbon dioxide) sensor 400-2 of the second mobile terminal 300-2 are configured to measure a concentration of the CO₂ (carbon dioxide) 510 in the identical determination target 500. The determination system 200 of the present example is different from the determination system 200 illustrated in FIG. 12 in the above described aspect.

In the present example, a radio intensity between the determination unit 10 and the wireless transmission unit 310-1 in the first mobile terminal 300-1 is set as a first radio intensity Sw1. In the present example, a radio intensity between the determination unit 10 and the wireless transmission unit 310-2 in the second mobile terminal 300-2 is set as a second radio intensity Sw2. The determination unit 10 may determine a state of the CO₂ (carbon dioxide) sensor 400 based on the first radio intensity Sw1 and the second radio intensity Sw2.

In the present example, since the CO₂ (carbon dioxide) sensor 400-1 and the CO₂ (carbon dioxide) sensor 400-2 measure the concentration of the CO₂ (carbon dioxide) 510 in the identical determination target 500, a probability is high that a ratio of a measured value of the concentration of the CO₂ (carbon dioxide) 510 by the CO₂ (carbon dioxide) sensor 400-1 to a measured value of the concentration of the CO₂ (carbon dioxide) 510 by the CO₂ (carbon dioxide) sensor 400-2 is matched within a predetermined error range with 1.0 as a center. Therefore, when the measured value of the concentration of the CO₂ (carbon dioxide) 510 by the CO₂ (carbon dioxide) sensor 400-1 and the measured value of the concentration of the CO₂ (carbon dioxide) 510 by the CO₂ (carbon dioxide) sensor 400-2 are out of the predetermined error range, a defect may have occurred in one of the CO₂ (carbon dioxide) sensors 400.

When a ratio (Sw1/Sw2) of the first radio intensity Sw1 to the second radio intensity Sw2 is lower than a predetermined value, the determination unit 10 may determine that the CO₂ (carbon dioxide) sensor 400-1 in the first mobile terminal 300-1 is faulty. The predetermined value is, for example, 0.1. When a difference (Sw2-Sw1) between the second radio intensity Sw2 and the first radio intensity Sw1 is larger than a predetermined difference, the determination unit 10 may determine that the CO₂ (carbon dioxide) sensor 400-1 in the first mobile terminal 300-1 is faulty.

The determination unit 10 may determine a location of the first mobile terminal 300-1 in the determination target 500 based on the first radio intensity Sw1, and determine whether the CO₂ (carbon dioxide) sensor 400-1 included in the first mobile terminal 300-1 is operative or faulty based on the determination result. The determination unit 10 may determine a location of the second mobile terminal 300-2 in the determination target 500 based on the second radio intensity Sw2, and determine whether the CO₂ (carbon dioxide) sensor 400-2 included in the second mobile terminal 300-2 is operative or faulty based on the determination result.

The determination unit 10 may determine the measurement situation Sm of the concentration of the CO₂ (carbon dioxide) 510 in the determination target 500 based on the first radio intensity Sw1 and the second radio intensity Sw2. The determination unit 10 may determine the measurement situation Sm based on the concentration of the CO₂ (carbon dioxide) 510, and the first radio intensity Sw1 and the second radio intensity Sw2. The determination unit 10 may determine the measurement situation Sm not based on the concentration of the CO₂ (carbon dioxide) 510, but based on the first radio intensity Sw1 and the second radio intensity Sw2.

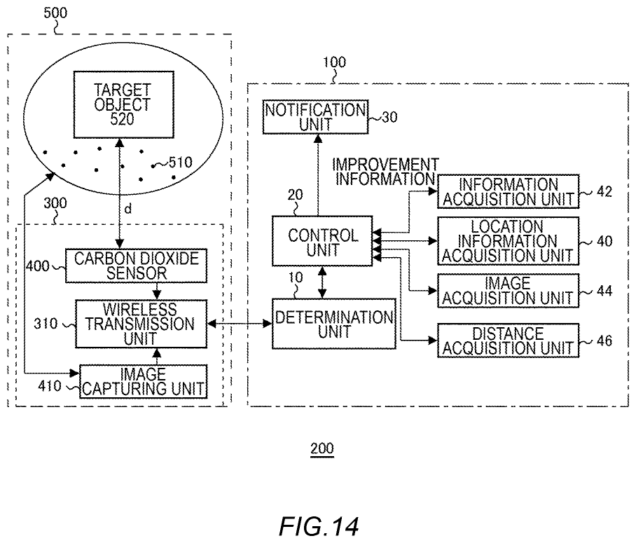

FIG. 14 is a block diagram illustrating another example of the determination system 200 according to one embodiment of the present invention. In the determination system 200 of the present example, the determination apparatus 100 further includes the distance acquisition unit 46. The determination system 200 of the present example is different from the determination system 200 illustrated in FIG. 7 in the above described aspect.

The distance acquisition unit 46 is configured to acquire the distance d between the CO₂ (carbon dioxide) sensor 400 and the target object 520. In the present example, the distance acquisition unit 46 acquires the distance d between the CO₂ (carbon dioxide) sensor 400 and the target object 520 based on the image acquired by the image acquisition unit 44. As described above, the target object 520 is a structural object in the determination target 500 which is a structural object that may affect at least one of the situation Sd and the measurement situation Sm of the CO₂ (carbon dioxide) concentration in the determination target 500. It should be noted that when the distance acquisition unit 46 is to acquire the distance d not based on the image acquired by the image acquisition unit 44, the distance acquisition unit 46 may acquire the distance d by an electro-optical distance meter or light detection and ranging (LiDAR).

When the distance d acquired by the distance acquisition unit 46 is shorter than a predetermined distance, the control unit 20 may control the notification unit 30 in such a way as to notify warning information on the notification unit 30. The warning information may be warning information indicating that the measurement situation Sm of the concentration of the CO₂ (carbon dioxide) 510 is inappropriate.

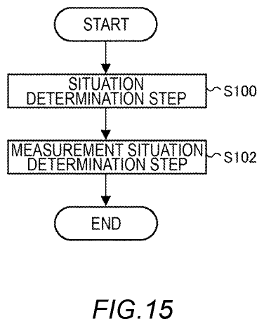

FIG. 15 is a flowchart illustrating one example of a determination method according to one embodiment of the present invention. The determination method according to one embodiment of the present invention will be described by using the determination system 200 illustrated in FIG. 1 is set as an example. A situation determination step S100 is a step where the determination unit 10 determines the situation Sd of the concentration of the CO₂ (carbon dioxide) 510 in the determination target 500 based on the concentration of the CO₂ (carbon dioxide) 510 in the determination target 500. A measurement situation determination step S102 is a step where the determination unit 10 determines the measurement situation Sm of the concentration of the CO₂ (carbon dioxide) 510 in the determination target 500 based on the concentration of the CO₂ (carbon dioxide) 510 in the determination target 500.

According to the determination method illustrated in FIG. 15, in the situation determination step S100, the determination unit 10 determines the situation Sd of the concentration of the CO₂ (carbon dioxide) 510 in the determination target 500, and in the measurement situation determination step S102, the determination unit 10 determines the measurement situation Sm of the concentration of the CO₂ (carbon dioxide) 510 in the determination target 500. Therefore, a user of the determination method can find out the situation Sd and the measurement situation Sm of the concentration of the CO₂ (carbon dioxide) 510 in the determination target 500.

It should be noted that the measurement situation determination step S102 may be performed after the situation determination step S100, or may also be performed in parallel with the situation determination step S100. The situation determination step S100 may also be performed after the measurement situation determination step S102.

The situation determination step S100 may be a step where the determination unit 10 determines the situation Sd of the concentration of the CO₂ (carbon dioxide) 510 based on the change over time of the concentration of the CO₂ (carbon dioxide) 510 in the determination target 500. With this configuration, the user of the determination method can find out the situation Sd determined based on the change over time of the concentration of the CO₂ (carbon dioxide) 510, and the measurement situation Sm of the concentration of the CO₂ (carbon dioxide) 510.

The situation determination step S100 may be a step where the determination unit 10 determines the situation Sd of the concentration of the CO₂ (carbon dioxide) 510 in the determination target 500 based on the concentration of the CO₂ (carbon dioxide) 510 in the determination target 500 and a concentration of CO₂ (carbon dioxide) 510 in another determination target 500 different from the determination target 500. A description will be provided while the determination system 200 illustrated in FIG. 12 is set as an example, and in the situation determination step S100, the determination unit 10 determines the situation Sd of the CO₂ (carbon dioxide) concentration based on the concentration of the CO₂ (carbon dioxide) 510-1 in the determination target 500-1 and the concentration of the CO₂ (carbon dioxide) 510-2 in the determination target 500-2. The determination target 500-1 and the determination target 500-2 are not interconnected.

In the situation determination step S100, the determination unit 10 determines the situation Sd of the CO₂ (carbon dioxide) concentration of one of the determination target 500-1 and the determination target 500-2. In the present example, when the determination unit 10 is to determine the situation Sd of the CO₂ (carbon dioxide) concentration in the determination target 500-1, the determination unit 10 compares the CO₂ (carbon dioxide) concentration of the determination target 500-1 with the CO₂ (carbon dioxide) concentration of the determination target 500-2 to determine the situation Sd of the CO₂ (carbon dioxide) concentration in the determination target 500-1. Therefore, the determination unit 10 more easily determines the more accurate situation Sd as compared with a case where the situation Sd of the CO₂ (carbon dioxide) concentration in the single determination target 500 is to be determined.

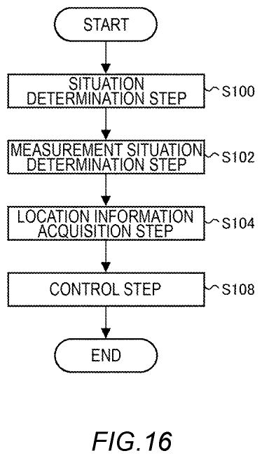

FIG. 16 is a flowchart illustrating another example of the determination method according to one embodiment of the present invention. The determination method of the present example is different from the determination method illustrated in FIG. 15 in an aspect that a location information acquisition step S104 and a control step S108 are further included. The determination method illustrated in FIG. 16 will be described while the determination system 200 illustrated in FIG. 2 is set as an example.

The location information acquisition step S104 is a step where the location information acquisition unit 40 acquires the location information of the determination target 500. The control step S108 is a step where the control unit 20 controls whether the determination result Rd by the determination unit 10 is to be notified by the notification unit 30 based on the location information of the determination target 500. As mentioned above in the description of FIG. 2, when the location information indicates a vicinity of a factory or the like where the concentration of the CO₂ (carbon dioxide) 510 is steadily high as compared with a location other than the vicinity of the factory, the control unit 20 may skip the notification of the determination result Rd by the determination unit 10 on the notification unit 30.

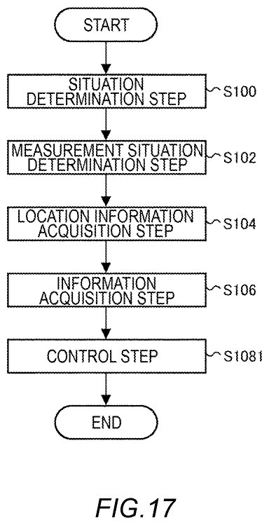

FIG. 17 is a flowchart illustrating another example of the determination method according to one embodiment of the present invention. The determination method of the present example is different from the determination method illustrated in FIG. 15 in an aspect that the location information acquisition step S104, an information acquisition step S106, and a control step S1081 are further included. The determination method illustrated in FIG. 17 will be described while the determination system 200 illustrated in FIG. 3 is set as an example.

The location information acquisition step S104 is a step where the location information acquisition unit 40 acquires the location information of the determination target 500. The information acquisition step S106 is a step where the information acquisition unit 42 acquires the improvement information Si according to at least one of the situation Sd of the concentration of the CO₂ (carbon dioxide) 510 and the measurement situation Sm of the concentration of the CO₂ (carbon dioxide) 510. The control step S1081 is a step where the control unit 20 controls the notification unit 30 in such a way as to notify the determination result Rd by the determination unit 10 and the improvement information Si on the notification unit 30 based on the location information of the determination target 500.

Since the determination method of the present example includes the control step S1081, when the situation Sd is, for example, a situation where the concentration of the CO₂ (carbon dioxide) 510 is an abnormal value, in the control step S1081, advertisement information or the like of one or more contractors who are close to the location of the determination target 500 corresponding to contractors who can eliminate the situation where the concentration of the CO₂ (carbon dioxide) 510 is an abnormal value is notified by the notification unit 30. With this configuration, the user of the determination method can find out the contractor who can eliminate the situation where the concentration of the CO₂ (carbon dioxide) 510 is an abnormal value.

A description will be provided while the determination system 200 illustrated in FIG. 5 is set as an example, and in the information acquisition step S106, the information acquisition unit 42 may further acquire the transmission destination information Is regarding the transmission destination to which the determination result Rd and the improvement information Si are to be transmitted. In the control step S1081, the control unit 20 may control the transmission unit 50 in such a way as to transmit the determination result Rd and the improvement information Si to the transmission destination based on the location information of the determination target 500. With this configuration, when the situation Sd is, for example, a situation where the concentration of the CO₂ (carbon dioxide) 510 is an abnormal value, in the control step S1081, the determination result Rd and the improvement information Si are transmitted to the contractor who can eliminate the situation where the concentration of the CO₂ (carbon dioxide) 510 is an abnormal value corresponding to the contractor who is the closest to the current location of the determination target 500.

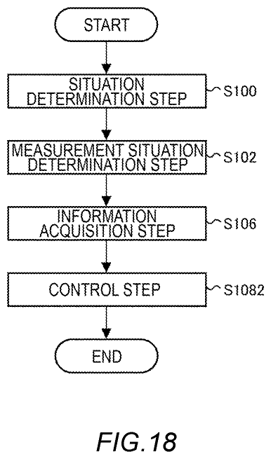

FIG. 18 is a flowchart illustrating another example of the determination method according to one embodiment of the present invention. The determination method of the present example is different from the determination method illustrated in FIG. 15 in an aspect that the information acquisition step S106 and a control step S1082 are further included. The determination method illustrated in FIG. 18 will be described while the determination system 200 illustrated in FIG. 6 is set as an example.

The information acquisition step S106 is a step where the information acquisition unit 42 acquires the improvement information Si according to at least one of the situation Sd of the concentration of the CO₂ (carbon dioxide) 510 and the measurement situation Sm of the concentration of the CO₂ (carbon dioxide) 510. The control step S1082 is a step where the control unit 20 controls the notification unit 30 in such a way as to notify the determination result Rd by the determination unit 10 and the improvement information Si on the notification unit 30. The control step S1082 may also be a step where the control unit 20 controls the notification unit 30 in such a way as to notify the determination result Rd by the determination unit 10 and the improvement information Si on the notification unit 30 based on the image acquired by the image acquisition unit 44.

According to determination method of the present example, in the control step S1082, the determination result Rd and the improvement information Si are notified by the notification unit 30. Therefore, the user of the determination apparatus 100 can find out the determination result Rd and the improvement information Si by visibly recognizing the notification unit 30.

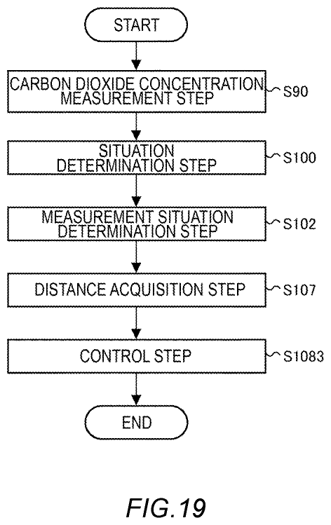

FIG. 19 is a flowchart illustrating another example of the determination method according to one embodiment of the present invention. The determination method of the present example is different from the determination method illustrated in FIG. 15 in an aspect that the carbon dioxide concentration measurement step S90, a distance acquisition step S107, and a control step S1083 are further included. The determination method illustrated in FIG. 19 will be described while the determination system 200 illustrated in FIG. 10 is set as an example

The carbon dioxide concentration measurement step S90 is a step where the CO₂ (carbon dioxide) sensor 400 measures the concentration of the CO₂ (carbon dioxide) 510 in the determination target 500. The distance acquisition step S107 is a step where the distance acquisition unit 46 acquires a distance between the CO₂ (carbon dioxide) sensor 400 and the target object 520. The distance acquisition step S107 may be a step where the distance acquisition unit 46 acquires a distance between the CO₂ (carbon dioxide) sensor 400 and the target object 520 based on the image acquired by the image acquisition unit 44. It should be noted that in the distance acquisition step S107, when the distance acquisition unit 46 is to acquire the distance d not based on the image acquired by the image acquisition unit 44, the distance acquisition step S107 may also be a step where the distance acquisition unit 46 acquires the distance d by an electro-optical distance meter or light detection and ranging (LiDAR).

The target object 520 may be a wall, a floor, a ceiling, a window, or a door of a structural object or a living body as described in the description of FIG. 10. The control step S1083 is a step where the control unit 20 controls the notification unit 30 in such a way as to notify warning information on the notification unit 30 when the distance acquired by the distance acquisition unit 46 is shorter than a predetermined distance. The warning information may be warning information indicating that the measurement situation Sm of the concentration of the CO₂ (carbon dioxide) 510 is inappropriate.

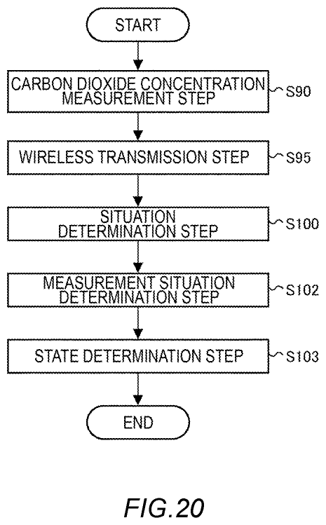

FIG. 20 is a flowchart illustrating another example of the determination method according to one embodiment of the present invention. The determination method of the present example is different from the determination method illustrated in FIG. 15 in an aspect that a wireless transmission step S95 and a state determination step S103 are further included. The determination method illustrated in FIG. 19 will be described while the determination system 200 illustrated in FIG. 13 is set as an example.

The wireless transmission step S95 is a step where the wireless transmission unit 310 included in each of the first mobile terminal 300-1 and the second mobile terminal 300-2 wirelessly transmits information of the concentration of the CO₂ (carbon dioxide) 510 measured by the CO₂ (carbon dioxide) sensor 400 in the carbon dioxide concentration measurement step S90 to the determination unit 10. The state determination step S103 is a step where the determination unit 10 determines a state of the CO₂ (carbon dioxide) sensor 400 based on the first radio intensity Sw1 between the wireless transmission unit 310 in the first mobile terminal 300-1 and the determination unit 10 and the second radio intensity Sw2 between the wireless transmission unit 310 in the second mobile terminal 300-2 and the determination unit 10.

The state determination step S103 may be a step where when a ratio of the first radio intensity Sw1 to the second radio intensity Sw2 is lower than a predetermined value or a difference between the second radio intensity Sw2 and the first radio intensity Sw1 is larger than a predetermined difference, the determination unit 10 determines that the CO₂ (carbon dioxide) sensor 400 of the first mobile terminal 300-1 is faulty.

Various embodiments of the present invention may be described with reference to a flowchart and a block diagram. According to the various embodiments of the present invention, a block may represent (1) a step of a process where operations are executed or (2) a section of an apparatus having a role for executing operations.

A specific step may be executed by a dedicated circuit, a programmable circuit, or a processor. A specific section may be implemented by a dedicated circuit, a programmable circuit, or a processor. The programmable circuit and the processor may be supplied together with a computer readable instruction. The computer readable instruction may be stored on a computer readable medium.

The dedicated circuit may include at least one of a digital hardware circuit and an analog hardware circuit. The dedicated circuit may include at least one of an integrated circuit (IC) and a discrete circuit. The programmable circuit may a hardware circuit including include logical AND, logical OR, logical XOR, logical NAND, logical NOR, and other logical operations. The programmable circuit may include a reconfigurable hardware circuit including a flip-flop, a register, a memory element such as a field programmable gate array (FPGA) and a programmable logic array (PLA), and the like.

The computer readable medium may include any tangible device capable of storing an instruction executed by an appropriate device. Since the computer readable medium includes the tangible device, the computer readable medium having the instruction stored on the device constitutes a product including an instruction that may be executed in order to provide means to execute an operation specified by a flowchart or a block diagram.

The computer readable medium may be, for example, an electronic storage medium, a magnetic storage medium, an optical storage medium, an electromagnetic storage medium, a semiconductor storage medium, or the like. More specifically, for example, the computer readable medium may be a floppy (registered trademark) disk, a diskette, a hard disk, a random access memory (RAM), a read only memory (ROM), an erasable programmable read only memory (EPROM or flash memory), an electrically erasable programmable read only memory (EEPROM), a static random access memory (SRAM), a compact disk read only memory (CD-ROM), a digital versatile disk (DVD), a Blu-ray (registered trademark) disk, a memory stick, an integrated circuit card, or the like.

The computer readable instruction may include any of an assembler instruction, an instruction-set-architecture (ISA) instruction, a machine instruction, a machine dependent instruction, a microcode, a firmware instruction, state-setting data, a source code, and a target object code. The source code and the object code may be written in any combination of one or more programming languages including an object oriented programming language and a procedural programming language in related art. The object oriented programming language may be, for example, Smalltalk (registered trademark), JAVA (registered trademark), C++, or the like. The procedural programming language may be, for example, a “C” programming language.

The computer readable instruction may be provided to a general purpose computer, a special purpose computer, or a processor or a programmable circuit of another programmable data processing apparatus locally or via a local area network (LAN) or a wide area network (WAN) such as the Internet. The general purpose computer, the special purpose computer, or the processor or the programmable circuit of the other programmable data processing apparatus may execute the computer readable instruction to provide means to execute operations specified by the flowcharts illustrated in FIG. 15 to FIG. 20 or the block diagrams illustrated in FIG. 1 to FIG. 14. The processor may be, for example, a computer processor, a processing unit, a microprocessor, a digital signal processor, a controller, a microcontroller, or the like.

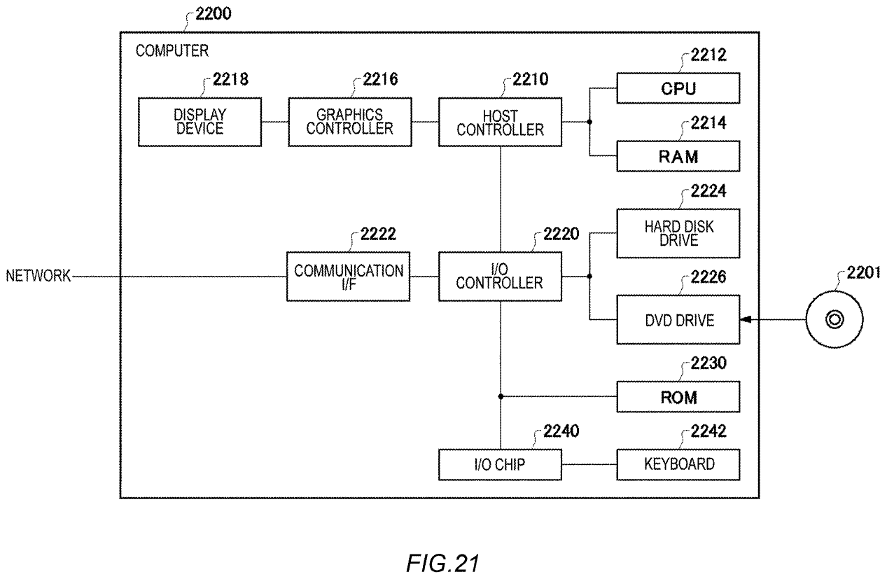

FIG. 21 illustrates one example of a computer 2200 through which the determination apparatus 100 according to one embodiment of the present invention may be entirely or partially embodied. A program that is installed in the computer 2200 can cause the computer 2200 to function as or perform operations associated with the determination apparatus 100 according to the embodiments of the present invention or one or more sections of the determination apparatus 100, or perform the operations or the one or more sections, or cause the computer 2200 to perform the respective steps (see FIG. 15 to FIG. 20) according to the determination method the present invention or steps of the processes. The program may be executed by the CPU 2212 to cause the computer 2200 to perform certain operations associated with some or all of the blocks of the flowcharts (FIG. 15 to FIG. 20) and the block diagrams (FIG. 1 to FIG. 14) which are described in the present specification.

The computer 2200 according to one embodiment of the present invention includes the CPU 2212, a RAM 2214, a graphics controller 2216, and a display device 2218. The CPU 2212, the RAM 2214, the graphics controller 2216, and the display device 2218 are mutually connected by a host controller 2210. The computer 2200 further includes input and output units such as a communication interface 2222, a hard disk drive 2224, a DVD-ROM drive 2226, and an IC card drive. The communication interface 2222, the hard disk drive 2224, the DVD-ROM drive 2226, and the IC card drive, and the like are connected to the host controller 2210 via an input and output controller 2220. The computer further includes legacy input and output units such as a ROM 2230 and a keyboard 2242. The ROM 2230, the keyboard 2242, and the like are connected to the input and output controller 2220 through an input and output chip 2240.

The CPU 2212 operates according to programs stored in the ROM 2230 and the RAM 2214, thereby controlling each unit. The graphics controller 2216 obtains image data generated by the CPU 2212 on a frame buffer or the like provided in the RAM 2214 or in the RAM 2214 itself to cause the image data to be displayed on the display device 2218.

The communication interface 2222 communicates with other electronic devices via a network. The hard disk drive 2224 stores programs and data used by the CPU 2212 within the computer 2200. The DVD-ROM drive 2226 reads the programs or the data from the DVD-ROM 2201, and provides the read programs or data to the hard disk drive 2224 via the RAM 2214. The IC card drive reads programs and data from an IC card, or writes programs and data to the IC card.

The ROM 2230 stores a boot program or the like executed by the computer 2200 at the time of activation, or a program depending on the hardware of the computer 2200. The input and output chip 2240 may connect various input and output units via a parallel port, a serial port, a keyboard port, a mouse port, or the like to the input and output controller 2220.

A program is provided by a computer readable media such as the DVD-ROM 2201 or the IC card. The program is read from the computer readable media, installed into the hard disk drive 2224, the RAM 2214, or the ROM 2230, which are also examples of the computer readable media, and executed by the CPU 2212. The information processing described in these programs is read into the computer 2200, resulting in cooperation between a program and the above-mentioned various types of hardware resources. An apparatus or method may be constituted by realizing the operation or processing of information in accordance with the usage of the computer 2200.

For example, when a communication is performed between the computer 2200 and an external device, the CPU 2212 may execute a communication program loaded onto the RAM 2214 to instruct communication processing to the communication interface 2222, based on the processing described in the communication program. The communication interface 2222, under control of the CPU 2212, reads transmission data stored on a transmission buffering region provided in a recording medium such as the RAM 2214, the hard disk drive 2224, the DVD-ROM 2201, or the IC card, and transmits the read transmission data to a network or writes reception data received from a network to a reception buffering region or the like provided on the recording medium.

The CPU 2212 may cause all or a necessary portion of a file or a database to be read into the RAM 2214, the file or the database having been stored in an external recording medium such as the hard disk drive 2224, the DVD-ROM drive 2226 (DVD-ROM 2201), the IC card, or the like. The CPU 2212 may perform various types of processing on the data on the RAM 2214. The CPU 2212 may then write back the processed data to the external recording medium.

Various types of information, such as various types of programs, data, tables, and databases, may be stored in the recording medium to undergo information processing. The CPU 2212 may perform various types of processing on the data read from the RAM 2214, which includes various types of operations, processing of information, condition judging, conditional branch, unconditional branch, search or replace of information, or the like, as described throughout the present disclosure and designated by an instruction sequence of programs. The CPU 2212 may write the result back to the RAM 2214.

The CPU 2212 may search for information in a file, a database, or the like in the recording medium. For example, when a plurality of entries, each having an attribute value of a first attribute associated with an attribute value of a second attribute, are stored in the recording medium, the CPU 2212 may search for an entry matching the condition whose attribute value of the first attribute is designated, from among the plurality of entries, read the attribute value of the second attribute stored in the entry, and read a second attribute value to obtain the attribute value of the second attribute associated with the first attribute satisfying the predetermined condition.

The above-explained program or software modules may be stored in the computer readable media on the computer 2200 or of the computer 2200. A recording medium such as a hard disk or a RAM provided in a server system connected to a dedicated communication network or the Internet can be used as the computer readable media. The program may be provided to the computer 2200 by the recording medium.

While the embodiments of the present invention have been described, the technical scope of the present invention is not limited to the above described embodiments. It is apparent to persons skilled in the art that various alterations and improvements can be added to the above described embodiments. It is also apparent from the scope of the claims that the embodiments added with such alterations or improvements can be included in the technical scope of the present invention.

The operations, procedures, steps, and stages of each process performed by an apparatus, system, program, and method shown in the claims, embodiments, or diagrams can be performed in any order as long as the order is not indicated by “prior to,” “before,” or the like and as long as the output from a previous process is not used in a later process. Even if the process flow is described using phrases such as “first” or “next” in the claims, embodiments, or diagrams, it does not necessarily mean that the process must be performed in this order.

### Supplementary Item 1

A determination apparatus including:

a determination unit configured to determine, based on a carbon dioxide concentration in a determination target, a situation of the carbon dioxide concentration in the determination target; and

a location information acquisition unit configured to acquire location information of the determination target,

in which

the determination unit is configured to further determine a measurement situation of the carbon dioxide concentration in the determination target based on the location information acquired by the location information acquisition unit.

### Supplementary Item 2

A determination apparatus including:

a determination unit configured to determine, based on a carbon dioxide concentration in a determination target, a situation of the carbon dioxide concentration in the determination target; and

an image acquisition unit configured to acquire an image of the determination target,

in which

the determination unit is configured to further determine a measurement situation of the carbon dioxide concentration in the determination target based on the image acquired by the image acquisition unit.

### Supplementary Item 3

A determination apparatus including:

a determination unit configured to determine, based on a carbon dioxide concentration in a determination target, a situation of the carbon dioxide concentration in the determination target; and

an environmental sound measurement unit configured to measure an environmental sound in the determination target,

in which

the determination unit is configured to further determine a measurement situation of the carbon dioxide concentration in the determination target based on the environmental sound measured by the environmental sound measurement unit.

### Supplementary Item 4

A determination system including:

a determination apparatus having a determination unit configured to determine, based on a carbon dioxide concentration in a determination target, a situation of the carbon dioxide concentration in the determination target; and

one or more mobile terminals, each having a carbon dioxide sensor configured to measure a carbon dioxide concentration in the determination target,

in which

each of the plurality of mobile terminals has a carbon dioxide sensor configured to measure a carbon dioxide concentration in the determination target, and a wireless transmission unit configured to wirelessly transmit information of the carbon dioxide concentration measured by the carbon dioxide sensor to the determination unit, and

the determination unit is configured to further determine a measurement situation of the carbon dioxide concentration in the determination target based on a first radio intensity between the wireless transmission unit in a first mobile terminal among the plurality of mobile terminals and the determination unit and a second radio intensity between the wireless transmission unit in a second mobile terminal among the plurality of mobile terminals and the determination unit.

### EXPLANATION OF REFERENCES

10: determination unit, 20: control unit, 30: notification unit, 32: sound output unit, 40: location information acquisition unit, 42: information acquisition unit, 43: storage unit, 44: image acquisition unit, 46: distance acquisition unit, 48: sound communication unit, 50: transmission unit, 52: transmission destination, 60: motion sensor, 62: environmental sound measurement unit, 100: determination apparatus, 200: determination system, 300: mobile terminal, 310: wireless transmission unit, 400: CO₂ (carbon dioxide) sensor, 410: image capturing unit, 500: determination target, 510: CO₂ (carbon dioxide), 520: target object, 2200: computer, 2201: DVD-ROM, 2210: host controller, 2212: CPU, 2214: RAM, 2216: graphics controller, 2218: display device, 2220: input and output controller, 2222: communication interface, 2224: hard disk drive, 2226: DVD-ROM drive, 2230: ROM, 2240: input and output chip, 2242: keyboard

What is claimed is:

1. A determination apparatus comprising:
a determination unit configured to determine, based on a carbon dioxide concentration in a determination target, a situation of the carbon dioxide concentration in the determination target and a measurement situation of the carbon dioxide concentration in the determination target;
a notification unit configured to notify a determination result by the determination unit;
an information acquisition unit configured to acquire improvement information according to at least one of a situation of the carbon dioxide concentration and a measurement situation of the carbon dioxide concentration; and
a control unit configured to control the notification unit to have the notification unit notify the determination result and the improvement information.

2. The determination apparatus according to claim 1, wherein the determination unit is configured to determine the situation of the carbon dioxide concentration based on a change over time of the carbon dioxide concentration.

3. The determination apparatus according to claim 1, wherein the determination unit is configured to determine the situation of the carbon dioxide concentration based on the carbon dioxide concentration in the determination target and a carbon dioxide concentration in another determination target different from the determination target.

4. The determination apparatus according to claim 1, further comprising
a location information acquisition unit configured to acquire location information of the determination target,
wherein the control unit is configured to control the notification unit so that the determination result and the improvement information is notified by the notification unit based on the location information of the determination target.

5. The determination apparatus according to claim 1, further comprising:
a motion sensor configured to detect at least one of an acceleration, an angular rate, and terrestrial magnetism information, wherein
the improvement information according to the measurement situation of the carbon dioxide concentration is information based on at least one of the acceleration, the angular rate, and the terrestrial magnetism information which are detected by the motion sensor.

6. The determination apparatus according to claim 1, further comprising:
a transmission unit configured to transmit the determination result and the improvement information, wherein
the information acquisition unit is configured to further acquire transmission destination information regarding a transmission destination to which the determination result and the improvement information are to be transmitted, and
the control unit is configured to control the transmission unit in such a way as to transmit the determination result and the improvement information to the transmission destination.

7. The determination apparatus according to claim 1, further comprising
an environmental sound measurement unit configured to measure an environmental sound in the determination target, wherein
the improvement information according to the measurement situation of the carbon dioxide concentration is information based on the environmental sound measured by the environmental sound measurement unit.

8. The determination apparatus according to claim 1, further comprising:
an image acquisition unit configured to acquire an image of the determination target, wherein
the determination unit is configured to correct the determination result based on the image acquired by the image acquisition unit.

9. The determination apparatus according to claim 8, wherein the determination unit is configured to correct the determination result when the image acquisition unit acquires an image of predetermined equipment that emits a predetermined amount or more of carbon dioxide.

10. The determination apparatus according to claim 1, further comprising:
a carbon dioxide sensor configured to measure a carbon dioxide concentration in the determination target;
a distance acquisition unit configured to acquire a distance between the carbon dioxide sensor and a target object,
wherein the control unit is configured to control the notification unit in such a way as to notify warning information on the notification unit when the distance acquired by the distance acquisition unit is shorter than a predetermined distance.

11. A determination system comprising:
the determination apparatus according to claim 1; and
one or a plurality of mobile terminals, each having a carbon dioxide sensor configured to measure a carbon dioxide concentration in the determination target.

12. The determination system according to claim 11, wherein
each of the plurality of mobile terminals has a carbon dioxide sensor configured to measure a carbon dioxide concentration in the determination target and a wireless transmission unit configured to wirelessly transmit information of the carbon dioxide concentration measured by the carbon dioxide sensor to the determination unit, and
the determination unit is configured to determine a state of the carbon dioxide sensor based on a first radio intensity between the wireless transmission unit in a first mobile terminal among the plurality of mobile terminals and the determination unit and a second radio intensity between the wireless transmission unit in a second mobile terminal among the plurality of mobile terminals and the determination unit.

13. A determination method comprising:
determining, by a determination unit, based on a carbon dioxide concentration in a determination target, a situation of the carbon dioxide concentration in the determination target;
determining, by the determination unit, a measurement situation of the carbon dioxide concentration in the determination target based on the carbon dioxide concentration in the determination target; and
acquiring, by an information acquisition unit, improvement information according to at least one of a situation of the carbon dioxide concentration and a measurement situation of the carbon dioxide concentration; and
controlling, by a control unit, a notification unit to notify a determination result by the determination unit and the improvement information.

14. The determination method according to claim 13, wherein the determining the situation is determining, by the determination unit, the situation of the carbon dioxide concentration based on a change over time of the carbon dioxide concentration in the determination target.

15. The determination method according to claim 13, wherein the determining the situation is determining, by the determination unit, the situation of the carbon dioxide concentration based on the carbon dioxide concentration in the determination target and a carbon dioxide concentration in another determination target different from the determination target.

16. The determination method according to claim 13, further comprising
acquiring, by a location information acquisition unit, location information of the determination target,
wherein the controlling, by the control unit, is controlling the notification unit in such a way as to notify the determination result by the determination unit and the improvement information on the notification unit based on the location information of the determination target.

17. The determination method according to claim 13, wherein
the acquiring the improvement information is further acquiring, by the information acquisition unit, transmission destination information regarding a transmission destination to which the determination result and the improvement information are to be transmitted, and
the controlling is controlling, by the control unit, a transmission unit in such a way as to transmit the determination result and the improvement information to the transmission destination based on location information of the determination target.

18. The determination method according to claim 13, further comprising:
measuring, by a carbon dioxide sensor, a carbon dioxide concentration in the determination target; and
acquiring, by a distance acquisition unit, a distance between the carbon dioxide sensor and a target object, wherein
the controlling, by a control unit, is controlling the notification unit in such a way as to notify warning information on the notification unit when the distance acquired by the distance acquisition unit is shorter than a predetermined distance.

19. The determination method according to claim 13, further comprising:
measuring, by a carbon dioxide sensor, a carbon dioxide concentration in the determination target;
wirelessly transmitting, by a wireless transmission unit included in each of a first mobile terminal and a second mobile terminal, information of the carbon dioxide concentration to the determination unit; and
determining, by the determination unit, a state of the carbon dioxide sensor based on a first radio intensity between the wireless transmission unit in the first mobile terminal and the determination unit and a second radio intensity between the wireless transmission unit in the second mobile terminal and the determination unit.

20. A computer readable medium having recorded thereon a program that, when executed by a computer, causes the computer to execute:
determining, based on a carbon dioxide concentration in a determination target, a situation of the carbon dioxide concentration in the determination target; and
determining a measurement situation of the carbon dioxide concentration in the determination target based on the carbon dioxide concentration in the determination target.
